(19) United States
(12) Patent Application Publication
(10) Pub. No.: US 2019/0117868 A1
(43) Pub. Date: Apr. 25, 2019

(54) Medical Device with an Optional Hydrophobic Filter Membrane and a Corresponding Front Support Structure

(71) Applicant: Fresenius Medical Care Deutschland GmbH, Bad Homburg (DE)

(72) Inventors: Manfred Weis, St. Wendel (DE); Martin Lauer, St. Wendel (DE)

(21) Appl. No.: 16/301,605

(22) PCT Filed: May 16, 2017
(86) PCT No.: PCT/EP2017/061734

§ 371 Date: Nov. 14, 2018

(30) Foreign Application Priority Data
May 19, 2016 (DE) 10 2016 109 196.0

(51) Int. Cl. A61M 1/30

(52) U.S. Cl. CPC A61M 1/304; A61M 2207/00; A61M 2205/7536; A61M 2205/126

(57) ABSTRACT

The present disclosure relates to a medical device, in particular a blood cassette, having a device body and having at least one fluid reception chamber for receiving at least one first medical fluid, in particular blood. The blood cassette can also have a hydrophobic filter device through which the fluid reception chamber is supplied with at least a second gaseous fluid, in particular air. The filter device comprises a filter membrane welded to the fluid reception chamber. The filter device also comprises a front support structure on or at front side, which is arranged for supporting the filter membrane.

### CROSS-REFERENCE TO RELATED APPLICATIONS

The present application is the national stage entry of International Patent Application No. PCT/EP2017/061734, filed on May 16, 2017, and claims priority to Application No. DE 10 2016 109 196.0, filed in the Federal Republic of Germany on May 19, 2016, the disclosures of which are incorporated herein in entirety by reference.

### TECHNICAL FIELD

The present disclosure relates to a medical device, in particular a blood cassette, and to a manufacturing method for the medical device.

### BACKGROUND

In order to be used for blood treatment, known blood treatment apparatuses are connected to a medical device, e.g. to a blood cassette, in which blood is treated or stored temporarily. Such blood cassettes are known from DE 10 2009 018 664 A1.

### SUMMARY

A medical device, in accordance with some embodiments described herein has a body, or another section of the medical device, that comprises at least a fluid system for a first medical fluid, in particular blood. The fluid system may be the device body partially or completely. The device body may be part of, or contribute to forming or establishing, the fluid system. The fluid system may comprise, of each, one or several channels, lines and/or fluid chambers.

The device body, or another section of the medical device, further comprises at least one, preferably hydrophobic, filter device. The filter device is configured and arranged such that a second, gaseous fluid, in particular air, can be supplied to the fluid system through the filter surface.

The filter device comprises at least one filter membrane. The filter membrane comprises a rear side and a front side.

The rear side may be the side of the filter membrane which faces away from an optional fluid reception chamber of the fluid system or an optional cover element (e.g. a film) of the medical device.

The rear side may be the side of the filter membrane on which no liquid is present during an intended use of the device.

The front side may be the side of the filter membrane which faces an optional fluid reception chamber of the fluid system or an optional cover element (e.g. a film) of the medical device.

The front side may be the side of the filter membrane on which there is a liquid or which faces a liquid during an intended use of the device.

The filter membrane is welded via its rear side, directly or indirectly, or on or at its rear side with the device body, e.g. with the optional fluid reception chamber, or a wall thereof. Thus, a weld section or a weld connection is established.

The filter device comprises, or is limited by, a support structure arranged on the front side of the filter membrane or in front of the filter membrane. Since the support structure is disposed on the front side of the filter membrane, in front of the front side or faces the front side, it is herein also referred to as front (alternative: front-sided) support structure.

The front support structure is preferably arranged for supporting the filter membrane, or it may in some usage or use situations serve this purpose. Supporting may be a limitation, holding, contacting, touching or the like. Supporting may be preventing an unallowed warpage or buckling of the filter membrane when or due to applying pressure on the rear side of the filter membrane.

A warpage may occur due to a pressure drop which is generated or which occurs with respect to the filter membrane. This may be achieved through a decrease of the pressure on the front side or through an increase of the pressure on the rear side of the filter membrane. The filter element may comprise a connection element for the connection with a line of a blood treatment apparatus, in which an excess pressure may be generated by a pump. This pump may be a compressor or an excess pressure reservoir. By means of said pump a liquid level can be reduced in the liquid chamber which contacts the filter membrane.

The use of “front” does not imply that there must be a “rear” support structure, although this can be the case in some exemplary embodiments.

The front support structure comprises a first contact section and contacts the latter with or through the weld section.

A method for manufacturing a medical device is described herein (in particular for manufacturing a medical device described herein).

The method serves for manufacturing a medical device (in particular a medical device described herein). It encompasses providing a device body of a medical device having a fluid system for a first fluid.

It further encompasses preparing a filter device having a filter surface through which a second gaseous fluid, in particular air, can be supplied. The filter device comprises at least one filter membrane, wherein the filter membrane has a rear side and a front side and wherein the filter membrane is welded at its rear side along a weld section with a section of the device body.

Furthermore, the method encompasses preparing a support structure or front support section.

The front support structure is arranged on the front side of the filter membrane at or on the device body. This is done such that the front support structure contacts the weld section through a first contact section of the front support structure.

Finally, the front support structure is welded to the front side of the filter membrane. The welding may be achieved by softening the material of the front support structure.

In all of the embodiments herein, the use of the expression “may be” or “may have” and so on, is to be understood synonymously with “preferably is” or “preferably has,” and so on respectively, and is intended to illustrate an exemplary embodiment.

Embodiments may comprise one or several of the aforementioned or following features. Thereby the features mentioned herein may in any combination be subject-matter of embodiments, unless the person skilled in the art recognizes their combination as technically impossible. Embodiments described herein are the subject-matter of the dependent claims as well.

Whenever numerical words are mentioned herein, the person skilled in the art shall recognize or understand them as indications of numerical lower limits. Unless it leads the person skilled in the art to an evident contradiction, the person skilled in the art shall comprehend the specification for example of “one” encompassing “at least one”. This understanding is also equally encompassed as the interpretation that a numeric word, for example, “one” may alternatively mean “exactly one”, wherever this is evidently technically possible for the person skilled in the art. Both can be encompassed by the embodiments described herein, and apply herein to all used numerical words.

The information “top” and “bottom” are to be understood herein in case of doubt by the person skilled in the art as absolute or relative spatial information which refer to the arrangement of the respective component during its use as intended.

The information “front” and “rear” and so on are to be understood herein in case of doubt by the person skilled in the art as absolute or relative spatial information which refer to the medical device. If the medical device comprises a cover element, the latter is thus disposed on the front side of the medical device.

A “contact” may be understood herein for example as a touching, a non-positive connection, a positive or fit-in connection, an adhesive bond or connection (e.g. by welding one to the other) or a mutual pressing between the relevant components or the components “contacting” each other, respectively.

In some exemplary embodiments, the medical device described herein can be embodied as a tubing system, a tubing set, a blood cassette or part of each, respectively.

The expression “fluid reception chamber” as presently used designates e.g. a chamber, or a part thereof, or a container, or a part thereof, having an interior or an inner space which is suited and, according to its purpose of use, intended for being completely or partly filled with fluids and for receiving the latter.

In some exemplary embodiments described herein, the fluid reception chamber comprises exactly two fluid inlets or fluid outlets. One of the two leads through the filter membrane and serves supplying and/or extracting gas, preferably only gas but not also liquid. The other one is the fluid connection for supplying and/or extracting liquid, e.g. from the first fluid.

A “first fluid” within the meaning of the present disclosure includes any medical liquid and/or any medical gas, as well as combinations thereof, which are envisioned or intended for introduction into a reception device as described herein. Preferably, the first fluid is blood.

The expression “first fluid” shall therefore be used synonymously with the expression “medical fluid.”

A “second fluid” is a gaseous fluid, or comprises gas, preferably air.

The expression “filter device” or “hydrophobic filter device”, as used synonymously herein, designates for example a device adapted and intended to exert a filter effect on fluids that are to be conducted through the fluid reception chamber. Alternatively, a filter device designates a membrane not having a filter effect.

The filter effect may encompass, e.g., a purification of the fluids being fed or supplied into the fluid reception chamber, in particular, for example, retaining of solids, micro-organisms such as viruses or bacteria, and the like.

The filter effect may also encompass that the filter is permeable for gas, but not for a liquid, in particular water or blood. With that, it is possible to prevent the liquid from penetrating into an area beyond the filter membrane. If, for example, the fluid chamber, which is limited by the filter membrane, is connected to the machine through an opening, the contamination of the machine may be prevented in case the liquid penetrates towards the filter membrane.

In some exemplary embodiments described herein, the front support structure comprises a second contact section. The front support structure is thereby arranged to contact, or it contacts, a support section of the device body by means of the second contact section.

In some exemplary embodiments described herein, the second contact section is welded to the support section of the device body.

In some exemplary embodiments described herein, the second contact section is glued to the support section of the device body.

In some exemplary embodiments described herein, the weld section and/or the first contact section are circumferential or closed sections or ring regions.

In some exemplary embodiments described herein, the second contact section is clamped with the support section of the device body.

For this, a clamping device may be provided.

The clamping device may be configured as flanging or clinch flange or may comprise the latter.

The clamping device may be circumferential or closed flanging or clinch flange sections.

In some exemplary embodiments described herein, the first contact section of the front support structure is made of a softer material than other sections or all other sections of the front support structure, or comprises a softer material than those sections.

Silicone is taken into account or considered as “softer” material (relative to other sections). As silicone is relatively expensive, it may be considered, for example, to manufacture only the first contact section from silicone. For other sections of the front support structure, for which the special characteristics of silicone are not needed or may even be disadvantageous (for example the high flexibility of components made of silicone is contrary to or contradicts the idea of support), a material may be selected or chosen which is stiffer or harder when compared to silicone or when compared to the material of the first contact section.

In some exemplary embodiments described herein, the first contact section of the front support structure is permanently made of softer material, in others, such material is only temporarily softer, for example when manufacturing or finishing up (through welding, by way of example), and, optionally, it becomes or gets harder again.

In some exemplary embodiments described herein, the front support structure is made of exactly one material or consists of exactly one material.

In some exemplary embodiments described herein, the first contact section of the front support structure is in—preferably direct—contact with the weld seam section of the device body. It is preferred that the weld seam section is in contact only with the first contact section.

In some exemplary embodiments described herein, the first contact section of the front support structure projects above or beyond the weld section of the device body in at least one direction or at least at one side of the weld section or it extends or stands over the weld section. The direction may be parallel to the main extension direction of the front support structure or the filter membrane. It is preferred that the projection or extension is towards the interior or central section of the filter membrane or the front support structure and/or towards one of the areas of the filter membranes which is, during use, pressurized or maximally loaded with pressure.

In some exemplary embodiments described herein, the first contact section of the front support structure is connected to the other sections of the front support structure in a releasable manner. The connection may be achieved by inserting or by an insertion.

In some exemplary embodiments described herein, the front support structure is, or consists of, a flat or planar grid.

In some exemplary embodiments described herein, the front support structure comprises openings.

In some exemplary embodiments described herein, the first contact section of the front support structure differs in color from further sections of the front support structure.

Hence, the first contact section may have e.g. a blue or another comparatively darker tone, while the front support structure is grey or white or transparent. In this way, the employee manufacturing the medical device or the doctor or staff responsible for using the medical device at a later point of time for treating the patient in the ward can check at a glance if the front support structure, which is usually manufactured separately from the device body, is indeed inserted into the device body. This facilitates the final check or inspection after completing the manufacturing. In addition this may contribute to the safety of the patient in that defective medical devices may still be detected or recognized at an early stage even at the patients' bedside.

In some exemplary embodiments described herein, the front support structure comprises openings. The diameter or width of the openings has a maximum of 0.7 mm, preferably a maximum of 0.5 mm. These dimensions have been proven to be favored in order to ensure a compromise between passage surfaces on the one hand and supporting the filter membrane on the other. The openings are larger than specified herein, then the filter membrane may, due to pressure, be or get pressed too deep into the openings. This may deteriorate a passage or permeability. Furthermore, the filter membrane may undesirably be heavily mechanically loaded. With these proposed dimensions of the openings, the filter membrane may be advantageously pressurized with at least 3 bar.

In some exemplary embodiments described herein, the front support structure comprises, or consists of, a second filter membrane.

In some exemplary embodiments described herein, the first contact section of the front support structure is, or comprises, a section which is formed during the manufacturing of the device by melting or temporarily liquefying of material. This melted section may be recognized as e.g. a flat material section which differs from the remaining structures of the front support structure wherein the latter are, when compared to said section, clearly defined, not melted and/or optically recognizable, differently manufactured or treated.

In some exemplary embodiments described herein, the front support structure comprises a profiled or laminated structure in its area facing the weld seam section. The profiling or lamination may to this end form or build channels extending parallel or diagonally to the main extension plane of the support structure. This combination of raised structures and channels, i.e. the profiling, may effect that when melting the structure for fastening the filter membrane to or on the front support structure, the material does not penetrate or only slightly penetrates into the central area of the filter membrane, but predominantly spreads or extends into or within the channel areas. This may protect the openings of the front support structure and the filter membrane against dumping or clogging due to or through melted material.

The filter device is arranged in some exemplary embodiments described herein such that a normal vector on the filter surface of the optional hydrophobic filter device does not extend parallel with a normal vector on the plane of the fluid level of the first fluid present in the fluid reception chamber during an intended use of the medical device and with a fluid reception chamber being filled more or less by the first fluid. In other words, the two normal vectors in these embodiments are not part of a common plane.

The expression “normal vector” as presently used in relation to the filter surface designates in some exemplary embodiments described herein a normal vector relative to an arbitrary surface portion or section or segment of the filter device. The normal vector on the filter surface may be perpendicular to a plane of a main section of the filter device, in a particularly preferred embodiment perpendicular to a surface of a main section of the filter device, in a quite particularly preferred embodiment a line perpendicular to a central section of the filter device or perpendicular to a filtering portion of the filter device or of the non-filtering membrane. Such a normal vector on the filter surface may represent a vertical plumb or a line perpendicular to one of the aforementioned portions.

The expression “normal vector on the plane of the fluid level” as used herein—in analogy with the definition for the normal vector on the filter surface as given in the foregoing—designates a normal vector relative to an arbitrary portion or segment or region of the plane of the fluid level.

The expression “fluid surface” relates to a fluid surface or level (these two expressions shall in the following be used synonymously) of a first fluid present or received in the fluid reception chamber for the use of the medical device.

In the event of hunting or sloshing, respectively, and/or flow movements, such as, for example rotating and/or undulating movements of the fluids present in the fluid reception chamber, it may be difficult to draw and determine a fluid level or a perpendicular line thereon, respectively. Accordingly, a fluid level is preferably understood herein to be an average level or filling height averaged under consideration of any sloshing and/or flow movements of the fluids present in the fluid reception chamber.

The fluid level may preferably be determined in a resting state of flow of the fluid or in static fluid conditions. It is insignificant whether such a resting state is obtained during use of the medical device. For the present purposes it is sufficient to assume or approximate such a resting state.

In some exemplary embodiments described herein, the normal vector on the filter surface is substantially perpendicular to the normal vector on the plane of the fluid level.

The expression “substantially perpendicular” as presently used encompasses deviations from right angles owing, e.g., to the fact that the medical device comprising the device exhibits a slight inclination during use, e.g. up to +/−15 degrees, whereas the fluid or liquid level in the medical device nevertheless remains horizontal at such an inclination of the filter device.

In some exemplary embodiments described herein, a normal vector on the filter surface does not have a point of intersection with a fluid level—as defined supra—of the first fluid present in the fluid reception chamber during use.

In some exemplary embodiments described herein, the medical device comprises at least one fluid supply chamber for supplying the second fluid.

The expression “fluid supply chamber” as used herein designates a chamber or a container having an interior or inner space suited and intended for receiving a second fluid and supplying it into the fluid reception chamber.

The fluid supply chamber may be manufactured as an injection-molded chamber. The fluid supply chamber may be a single-use fluid supply chamber.

The fluid supply chamber may be connected to the fluid reception chamber in at least one portion thereof.

It may be connected to the fluid reception chamber by material connection or may be integrated therewith.

The fluid supply chamber may have been formed, for example, during manufacture of the fluid reception chamber.

The fluid supply chamber may be separate from the fluid reception chamber by at least one partition or partial wall.

In some exemplary embodiments described herein, the filter device is disposed in an interior of the fluid supply chamber.

Preferably, the fluid supply chamber and the fluid reception chamber are in fluid communication with each other via the filter device alone exclusively.

The second fluid is preferably introduced or supplied or fed from the fluid supply chamber through the filter device into the fluid reception chamber during use of the medical device.

In a particularly preferred embodiment described herein, the filter device is configured and provided such that a first fluid and in particular a liquid fluid present in the fluid reception chamber cannot enter the fluid supply chamber via the filter device.

In some exemplary embodiments described herein, the second fluid may be supplied, made to flow, introduced, etc. into the fluid supply chamber via a fluid connector which is connectable, or connected, to the fluid supply chamber.

The fluid connector may be a sleeve-type component. It may be manufactured integrally with a wall or side wall of the fluid supply chamber. It may be provided on or at an outer face of the wall or side wall of the fluid supply chamber. It may be provided inside a region of the filter device disposed in the fluid supply chamber. It may be directly connected or coupled to the filter device.

The fluid connector may be connected to an inside of the wall or side wall of the fluid supply chamber through the intermediary of one or several connecting bores.

The fluid connector may be arranged to directly lead or conduct a fluid from an exterior of the medical device into an interior.

In some exemplary embodiments described herein, the fluid reception chamber may be manufactured for example in the form of an injection-molded chamber.

The fluid reception chamber may be a single-use fluid reception chamber.

The fluid reception chamber may be in a fluid communication with an outside of the chamber. It may have two or more fluid communications with the outside of the chamber.

The fluid reception chamber may have a fluid or exactly one fluid communication with an interior of the chamber only. It may have two or more fluid communications with the inside of the chamber.

By means of the filter membrane, the fluid reception chamber is preferably uncoupled from the fluid connector of the fluid supply chamber and/or from an outside of the medical device, regarding a liquid communication.

The filter membrane may have any suitable shape. It may be configured, for example, to be round, polygonal, in particular rectangular, elliptical, and the like.

The filter membrane may be cut out from a filter membrane strip or tape and/or may—where appropriate—be cut to a particular shape.

The filter membrane may be a single-use filter membrane.

The filter membrane may be a hydrophobic membrane. The filter membrane may be hydrophobic on at least one side.

The filter membrane may consist of two layers, i.e., firstly the proper membrane itself mostly consisting of a material that is difficult to weld and difficult to glue such as, e.g., PTFE (polytetrafluoroethylene) or ePTFE (expanded Polytetrafluoroethylene), and secondly a layer having drainage and/or support function and mostly consisting of, or comprising, a woven and/or nonwoven material suitable for being welded and/or glued. Apart from the aforementioned layers, the filter membrane may alternatively comprise additional layers or components.

The filter membrane may be a sterile membrane.

The filter membrane may be consist of, or comprise, a material which is not or ill-suited for gluing, and/or a material which is not or ill-suited for welding.

During use of the medical device such as, for example, during the duration of a treatment, the filter membrane may be exposed to varying pressures from the direction of the fluid reception chamber (outflowing gas, liquid in contact) and/or from the direction of a treatment apparatus (inflowing gas, liquid in contact in case of fault and/or undesirable liquid in contact resulting from condensation). As a protection of the filter membrane in the planar area of utilization and/or at its locations of sealing (generally welding), e.g. on walls of the fluid supply chamber, against a deformation by these pressure differences which might cause a structural damage or a tear in the membrane, a mechanically supporting the filter membrane may be advantageous.

In order to achieve such a mechanical support it is possible to employ a support structure. The support structure is configured such that the support surfaces do not seal the filter membrane against the desired passage of fluid through the filter membrane, e.g., a passage of useful gas, where this is not desirable.

The filter device may have a symmetrical or an asymmetrical construction.

In some exemplary embodiments described herein, the filter device comprises a support structure at or on both sides, i.e. both on the front side and on the rear side of the filter membrane.

In addition to the front support structure, a support structure, designated herein as “rear” support structure may be provided on the rear side of the filter membrane. These two support structures may be provided separately.

A third support structure may be arranged on the membrane layer itself. It may preferably have the form of a nonwoven or woven material, or the like.

The rear support structure is preferably provided inside the housing of the fluid connector. It may have a drainage structure. The rear support structure may have a drainage effect.

The rear support structure may be connected by non-positive and/or by positive connection and/or may be bonded to the fluid supply chamber. Preferably, the rear support structure is connected in an outer peripheral region by non-positive and/or by positive connection and/or may be bonded to the fluid supply chamber or to another section of the device body.

As the filter membrane generally consists of a material that is not or ill-suited for bonding and/or not or ill-suited for welding, the support structure facing the interior of the fluid supply chamber may be connected to the fluid supply chamber in at least one outer region, preferably an outer peripheral region thereof.

Preferably, the rear support structure is bonded to the fluid supply chamber or to another section of the device body, for example by means of welding as, e.g., by means of thermal welding, mirror welding, ultrasonic welding or laser welding. The rear support structure may be thermally welded to the fluid supply chamber or to a connecting region thereof.

To this end, the rear support structure is—preferably in an outer region thereof—made of a higher-melting material than the connecting region of the fluid supply chamber.

During welding the rear support structure with the fluid supply chamber or a connecting region thereof, the material of the fluid supply chamber liquefied by heating may penetrate into a porous structure of the rear support structure. The liquid material may penetrate up to the filter membrane. In this way, it is advantageously possible to form a non-releasable connection between the fluid supply chamber, the rear support structure, and the filter membrane. At the same time, the filter membrane may advantageously be sealed, in particular in a lateral direction.

The rear support structure is preferably a thin-walled injection-molded part. It preferably comprises a drainage structure on the side facing the membrane layer or on both sides.

The rear and/or front support structure may be produced or manufactured by means of injection molding.

For example, the rear support structure may be integrated in a wall of the fluid supply chamber manufactured by an injection molding technique.

The front support structure may be disposed substantially in parallel with the filter membrane.

The front support structure may be provided in force-free contact with the filter membrane and/or with little play on the side of the filter membrane facing away from the interior of the fluid supply chamber or may be in contact with the filter membrane.

The front support structure may be made of, or may comprise, the same material as the fluid supply chamber.

The front support structure may form, or maybe manufactured as, a separate element. For instance, the front support structure may be manufactured as a thin-walled injection-molded part.

In some exemplary embodiments described herein, the front support structure covers the filter membrane substantially completely.

The front support structure may have its area and/or boundary delimited towards the outside by a ring zone or a ring region which does not have a drainage effect.

The expressions “ring zone” or “ring region” designate in some exemplary embodiments an external region or external margin or an external edge or outer margin or outer edge of the front support structure. The word component “ring” may be a circular configuration of the zone or of the region. Alternatively, the “ring” may be a peripheral or circumferential region or a peripheral or circumferential zone which may be configured in any other suitable form, for example in the shape of a rectangle, an ellipse, and the like.

An outer boundary of this ring zone or ring region may substantially correspond to the external dimensions of the filter membrane.

An internal boundary of this ring zone or ring region may substantially correspond to the filter membrane area having a filtering effect which is left after fixation of the filter membrane to the ring zone or ring region.

The filter membrane may be connected to the front support structure in a gas-tight manner. It may be connected to a wall material of the ring zone or ring region. For instance, the filter membrane may be connected in an outer, preferably peripheral or circumferential, region thereof to the front support structure or to an outer ring region of the latter.

The filter membrane may be bonded to the wall material. It may, for example, be glued to or welded to the wall material. Welding methods include, inter alia, thermal welding, mirror welding, ultrasonic welding or laser welding.

The filter membrane may correspondingly be connected to or integrated with both the rear support structure and the front support structure.

The front support structure may be a thin-walled injection-molded part or element that is connectable to the filter membrane and/or to the device body by means of welding and/or gluing.

The front support structure may be welded by means of, inter alia, thermal welding, mirror welding, ultrasonic welding or laser welding.

A construction comprising at least three support structures may exemplarily be formed as follows:

The third support layer is applied on the filter membrane. During assembly, the third support layer, e.g. a weldable nonwoven material, is placed on the rear support structure on the side of the nonwoven material. The third support layer is welded with and/or glued to the rear support structure in a peripherally or circumferentially sealing manner at the circumference of the filter membrane layer, i.e., preferably in the region situated outside the rear support structure. In this way, a sealing function between the third support structure and the rear support structure may advantageously be achieved.

The front support structure, e.g., a thin-walled injection-molded part, is placed like a lid with its support structure on the filter membrane which is optionally welded to the rear support structure. It is welded to and/or bonded to the housing (injection-molded housing) of the fluid connector in an outer ring zone outside the filter membrane layer.

The front support structure advantageously provides a retaining function for the filter membrane.

The rear and/or front support structure(s) may be configured such that after arranging the support structure(s) there are still some freely accessible surface parts of the filter membrane present without any support, wherein each of the freely accessible surface parts have a sufficiently small extension to the adjacent mechanical supports or support structures.

Thus, the maximum admissible fluid pressure of the second fluid acting on the unsupported filter membrane areas preferably does not generate an inadmissibly high stress (for instance due to buckling of the filter membrane) any more.

For example, single ones or all of the support structures such as, for example, drainage structures, may have a width of about 0.5 to 2 mm.

The fluid connector arranged on the outside may communicate via bores, recesses or openings with the outer drainage or support structure, i.e., the rear support structure, for the filter membrane.

The filter membrane arranged between the rear and front support structures can substantially endure a mechanical load up to such an extent that is required in accordance with the purpose of use.

The plane in which such a membrane connection—i.e., a connection between the filter membrane and the two support structures—is arranged may substantially correspond to the plane in which projected regions of the drainage structure are arranged.

Depending on the connection technique and/or thickness of the filter membrane, a staggered height between projected regions of the drainage structure and the outer-side plane of the filter membrane may be reasonable. Such a staggered height may serve to let the outer-side plane of the filter membrane rest on the projected drainage structures in force-free contact and/or with little play.

The projected drainage structures may be arranged to be as small as possible.

Preferably only a restricted flow of the second fluid may take place through the filter membrane regions that come into contact and/or in pressed contact with the projected drainage structures that rest against them.

The size and/or number of the connecting bores to the fluid connector and/or the arrangement, number, width and/or depth of the recessed drainage structures may be such that a possible pressure drop of the second fluid caused by these flow paths makes up for a negligible or acceptable fraction of the total pressure drop occurring upon passage through the filter device.

The width of the recessed drainage structures and/or the diameters of the connecting bores to the fluid connector may be adapted to be sufficiently small such that the tensile forces acting on the filter membrane under maximum possible pressure differences (resulting, for example, in buckling into the recessed structures) are clearly lower than the admissible tensile forces, preferably both within the filter membrane and in the—generally more sensitive—zones at the interface to the ring fixation (e.g., weld).

In some exemplary embodiments described herein, the rear and front support structures comprise drainage structures that are identical in a mirror-reversed manner relative to a main plane of the membrane, or substantially identical.

Preferably, the recessed and/or projected drainage structures oppose each other in a congruous or substantially congruous manner.

Preferably, the recessed drainage structures on the one filter membrane side are realized to be narrower, or in turn the projected drainage structures on this side of the filter membrane are realized to be wider than the drainage structures on the other side of the filter membrane. This may allow a greater lateral installation tolerance. The pitches and/or arrangements of the structures may, however, in a preferred manner be realized identically on both sides.

In this way, by making use of the lateral installation tolerances, a very constant property profile concerning properties such as fluid passage resistance and degree of mechanical support may result.

The overall thickness of the structure of the front support structure may result from the depth of the drainage structure and/or from the minimum possible wall thickness of the material of the front support structure, or be the sum thereof. As a result, the front support structure may advantageously require little structural space and/or be manufactured at lower cost.

Another advantage may arise from the fact that there are no particular demands to the front support structure with regard to accuracy and rigidity. It may moreover advantageously be possible to fasten the front support structure at the fluid supply chamber solely under aspects of costs and/or the lowest possible complexity.

As is shown in FIGS. 1 and 2, the front support structure may, for example, be connected to the fluid supply chamber by means of plug-in or riveting fixation or according to the principle of bolt fixation. Likewise, the front support structure may be connected to the fluid supply chamber by means of dot-shaped welds and/or snapping into suitable geometrical configurations of a side wall or wall of the fluid supply chamber.

In some exemplary embodiments described herein, the drainage structures of the front support structure may differ from the outside drainage structures in that the former do not end at the membrane boundaries at their outside but radially continue up to the component boundary at their outside. Thus, fluids flowing in and/or out may freely penetrate into the remaining annular or ring space between the front support structure and an upper boundary or an upper margin or edge of the fluid supply chamber. The fluids may advantageously communicate with the fluid reception chamber via a large lumen.

In some exemplary embodiments described herein, the fluid reception chamber has a first structural height, and the fluid supply chamber has a second structural height different from the first structural height.

Also, or in addition, the fluid supply chamber may be disposed above the fluid reception chamber (“top”) during use of the medical device. “Above” may relate to a reference system passing through the center of the Earth.

In such an arrangement of the fluid reception chamber and the fluid supply chamber, the medical device is preferably embodied with a stepped depth. The deep fluid reception chamber arranged below the fluid supply chamber (“bottom”) during use of the medical device may be utilized as a reservoir and/or treatment space for the fluids present therein during use.

The medical device may be provided with a cover element on at least one side.

In a particularly preferred embodiment described herein, the filter means and/or the filter membrane are arranged in parallel or substantially in parallel with the cover element of the medical device.

In some exemplary embodiments described herein, the second fluid is a gas. In a further preferred embodiment, the first fluid may be a liquid such as, for example, blood.

The medical device described herein is suited for use in, or on, or with a treatment apparatus such as a medical treatment apparatus, an apparatus used in laboratory technology, an apparatus used in food and/or drug manufacture. Fluids suited for being introduced or supplied or conducted into the reception means may therefore encompass both medical liquids such as blood, substituate (e.g., saline solution), preparations of active agents such as solutions, suspensions, emulsions, carrier gases for active agents, cleaning liquids or gases, disinfection liquids or gases, sterilization liquids or gases, beverage liquids, and the like.

When the filter membrane is provided as a sterile membrane, the medical device may in particular be utilized for sterile air supply to the fluid reception chamber.

A medical device as described herein may be a single-use component or a single-use article manufactured, e.g., of a plastic material.

The medical device as described herein may be manufactured by means of an injection molding technique.

The medical device as described herein may include liquid and/or gas connections, semi-open passages and/or chambers and/or structures for coupling to actors and/or sensors. Such actors and/or sensors may serve for performing functions preferably non-invasive and/or uncoupled as regards sterility on the liquids in the cassette. One or several cover members such as, for example, membranes, in particular low-cost films, may provide for closure and/or sealing of the passages and chambers.

In certain exemplary embodiments described herein, the filter device or the filter membrane of the medical device are arranged in parallel with a cover element of the medical device for closing an interior of the fluid reception chamber against an outside.

For example, the blood treatment for which the medical device is utilized may be a dialysis method, hemodialysis, hemofiltration, hemodiafiltration and the like.

The medical device as described herein, may advantageously be utilized for a sterile air supply to a fluid reception chamber.

In certain exemplary embodiments described herein, the fluid supply chamber and the fluid reception chamber have together only exactly two connections to an exterior outside of these two chambers.

In certain exemplary embodiments described herein, the filter membrane is not arranged in a tubing section.

A tubing may be a flexible line for a fluid along its entire cross section.

In certain exemplary embodiments described herein, there is no tubing connections other than the fluid supply chamber and the fluid reception chamber.

In certain exemplary embodiments described herein, neither the fluid supply chamber nor the fluid reception chamber are flexible or completely flexible bags.

In certain exemplary embodiments described herein, the medical device is not a device for the selective blocking of the passage of fats.

In certain exemplary embodiments described herein, the medical device, the fluid supply chamber and/or the fluid reception chamber comprises no insertion pin.

In certain exemplary embodiments described herein, the access for the second fluid to the fluid supply chamber is on one side of the filter membrane (relative to a flow path through the filter membrane) and the access for the first fluid to the fluid reception chamber is on the other side thereof.

In certain exemplary embodiments described herein, the fluid reception chamber is directly connected to a fluid channel of the medical device, wherein the fluid channel extends through or is formed by, at least in sections, the device body.

In certain exemplary embodiments described herein, the fluid supply chamber is fluidly limited by the filter membrane.

In certain exemplary embodiments described herein, the fluid supply chamber and the fluid reception chamber are provided in the device body of the medical device.

In certain exemplary embodiments described herein, the medical device is a blood cassette. The device body is in these cases a cassette body.

In certain exemplary embodiments described herein, the medical device further comprises at least two connectors for pump tubing segments for peristaltic pumps, with or without pump tubing segments.

In certain exemplary embodiments described herein, the medical device comprises in addition an arterial patient connection, an arterial filter line, a venous patient connection and an arterial heparin addition site. The arterial heparin addition site is arranged between the arterial filter line and the venous patient connection.

In certain exemplary embodiments described herein, the medical device comprises a venous filter line and a venous heparin addition site, wherein the venous filter line is arranged medially to the venous heparin addition site.

In certain exemplary embodiments described herein, the medical device comprises a single needle sterile membrane which has the form of a parallelogram.

Using the filter membrane as single-needle sterile membrane poses a particular requirement on said filter membrane as a pressure from the rear side of the filter membrane is repeatedly built up in order to supply the blood of the fluid system to the patient. Such function, the filter membrane may effect a protection of the machine by preventing a penetration of liquid into the machine and a protection of the patient by preventing a contamination of the fluid system.

In a preferred embodiment described herein, the medical device comprises a primary or first alignment center and a secondary or second alignment center, e.g. in the shape of centering opening. The primary alignment center and the secondary alignment center comprise different forms and/or different alignments.

Some or all of the embodiments described herein may encompass one or several advantages mentioned supra or in the following.

The medical device as described herein may, due to its front support structure described herein, be sterilized in some embodiments by radiation sterilization or by “e-beam”. The radiation sterilization and “e-beam” may lead to a delamination of the filter membrane which is usually made of a thin Teflon layer and a thick carrier fleece. Thus, the resistance or firmness of the filter membrane against pressure is decreased. If the pressure exceeds a relatively low value, the Teflon layer may come off the carrier fleece (or vice versa), which may be detrimental to both the safety of the patient and also to the function of the filter membrane or of the medical device and is referred to herein as delamination. The delamination occurs preferably at the weld section on which the filter membrane is welded to the device body. The front support structure is, in contact with the weld section. It is this contact which may counteract a delamination by a given pressure or which may allow a pressure load, during use, with a higher pressure by a factor of 4 without the occurrence of delamination. It is thereby significant that the support of the filter membrane is achieved at its front side.

It is further advantageous when the contact between the first support section and the filter membrane extends beyond the weld section to an interior of the filter membrane.

In contrast with conventional arrangements for the sterile air supply of external functional devices in which the hydrophobic sterile membranes are disposed at the highest point of the fluid reception chamber and substantially in parallel with the free fluid surface, the filter membrane provided may preferably be arranged in a geodetic manner above the fluid reception chamber that is normally filled with fluids to the maximum. During failure-free operation, the fluid supply chamber may therefore advantageously only get into contact with the second fluid, for example gas, and with small quantities of the first fluid, e.g., a liquid such as blood.

The filter membranes used may be embodied as single-needle filter membranes.

The filter membranes used may have the form of a parallelogram.

As the filter membranes used are sensitive elements and difficult to examine and should therefore generally be replaced for every treatment utilization, the filter membrane may advantageously be a component of the single-use medical device. Such filter membranes may moreover in a further advantageous manner be sterilized in combination with the single-use medical device and/or keep the single-use partial system closed in a sterile manner during storage and/or during connecting to the treatment apparatus.

Hydrophobic filter membranes may furthermore advantageously ensure that the fluids to be treated which are present in the fluid reception chamber are prevented from entering the treatment apparatus in the event of malfunction.

The medical device as described herein may advantageously utilize the spatial and/or functional arrangements present in single-use medical device frequently provided for treatment apparatuses, in particular blood treatment apparatuses, for accommodation, for pressing, for compensating tolerances, for limiting forces, for mounting, for orientation and/or for handling in parallel with other functional units of the overall arrangement.

The different geometrical environmental conditions of conventional arrangements together with the arrangement of the fluid supply chamber and of the fluid reception chamber of the medical device may advantageously result in a higher gas pressure loss per area unit at the drainage or support structures due to confined space in the surroundings of the filter membrane. In this way, it may advantageously be possible to employ more cost-efficient and smaller-structured filter membranes at an identical pressure loss, so that it is at the same time frequently possible to utilize the structural space more efficiently due to the rectangular shape.

While the filter membrane in conventional systems generally has to be made round for reasons of mechanical strength of the surrounding supporting construction and has to be fitted into the housing environment at low lateral tolerances, the filter membrane in the device may be manufactures as a rectangle from a filter membrane without cutting losses and may be welded, bonded or pressed onto the wall of the fluid supply chamber with amply lateral tolerances.

In contrast with welded connections between connection mates that are of a same type or melted on both sides, which would result in the same sealing effect between the support structures and the filter membrane but would be accompanied by a lower mechanical susceptibility of the connection to strain, the selection of a low-melting material for the fluid supply chamber allows to achieve a sealing connection which may at the same time well be subjected to mechanical strains. In this way, it is advantageously possible to avoid the first support layer becoming thinner due to melting and pressing. Furthermore it is advantageously possible to avoid material faults and/or unfavorable discontinuities of cross-section at the interfaces to the non-molten regions of the filter membrane.

Good accessibility of the filter membrane from a lateral direction in the plane of the filter membrane, is moreover possible with the front support structure provided. This may be a particular advantage in the event of inadvertent wetting of the filter membrane with liquid.

The higher drainage capability towards the interior of the fluid reception chamber may advantageously result in important safety advantages: In conventional filter membrane arrangements when filter membranes are arranged in parallel with the surface of the liquid, a pressure shock may occur in the event of inadmissible complete flooding up to the filter membrane, which may lead both to a destruction of the filter membrane and interference with other components of the medical device and/or of the treatment apparatus. With the medical device as described herein, however, a level inadmissibly continuing to rise may sweep continuously across the filter membrane surface, so that the passage pressure may rise in a gently increasing manner and without shocks.

In addition, the arrangement of the filter means provided as described herein may advantageously endure innocuous multiple repeatability of a malfunction involving complete flooding of the fluid reception chamber, as the liquid is under the influence of gravity, capable of automatically draining again from the filter membrane into the fluid reception chamber.

In this way, it may advantageously be possible to avoid an abortion of the treatment method.

In the following, the present invention shall be described by making reference to the appended drawings. In the drawings, identical reference numerals designate same or identical elements, wherein:

### BRIEF DESCRIPTION OF THE FIGURES

FIG. 1 is a top view of a detail or section of the medical device as described herein, exemplarily configured as a blood cassette;

FIG. 2 is a cross-sectional view of a blood cassette as described herein;

FIG. 3 is an enlarged detail of the blood cassette shown in FIG. 2, shown in a longitudinal sectional view and tilted by 90° relative to FIG. 2 and relative to an arrangement during use of the blood cassette;

FIG. 4a is a view of a front support structure with a first contact section;

FIG. 4b is an enlarged view of details of the front support structure which is shown in FIG. 4a;

FIG. 5a-5d are highly simplified views of details of the blood cassette as described herein in different, exemplary embodiments;

FIG. 6a-6c are views of sections of both the cassette body and the front support structure in different, exemplary embodiments of the blood cassette;

FIG. 7a-7d are views of different embodiments of the second contact section and of the support section of the blood cassette as described herein;

FIG. 8 is a view of a device as described herein with a second filter membrane which is provided instead of the front support structure;

FIG. 9 is, like in FIG. 8, a view of an embodiment with a second filter membrane;

FIG. 10 is a view of a further embodiment as described herein of the front support structure prior to a melting of sections thereof;

FIG. 11 is a view of a further embodiment as described herein of the front support structure after a melting of sections thereof; and

FIG. 11a is a view of an enlarged detail of FIG. 11.

### DETAILED DESCRIPTION OF THE FIGURES

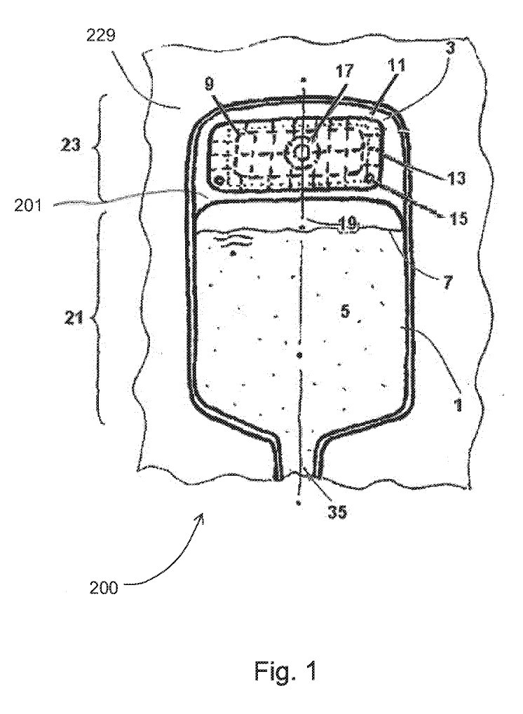

FIG. 1 shows a top view of a detail of a medical device as described herein, embodied as a blood cassette 200 according to an exemplary embodiment.

Although the medical device as described herein is herein exemplarily embodied as a blood cassette 200 and although it is being discussed or elaborated on in this regard, the medical device shall not be limited thereto. The medical device as described herein may by way of example be a tubing system, a tubing set or (a) part of each.

The blood cassette 200 comprises a cassette body 201. The blood cassette 200 further comprises, optionally, a cover element 229, herein a film. The cover element 229, if provided, covers the cassette body 201 at least partially, possibly at an entire side thereof. The cover element 229 is made of a softer material than the cassette body 201 for which reason the latter is referred to as hard part. The cover element 229 covers the cassette body 201 against an exterior or an atmosphere.

The cassette body 201 comprises a fluid reception chamber 1 as well as an optional fluid supply chamber 3.

During use, there is a first medical fluid 5 temporarily present in the fluid reception chamber 1 which fluid 5 fills the fluid reception chamber 1 in FIG. 1 up to a fluid level 7.

The fluid supply chamber 3 which optionally comprises a filter device, of which a filter membrane 9 as well as a likewise optional rear support structure 11 are shown in FIG. 1.

The rear support structure 11 comprises a drainage structure 13. The rear support structure 11 is connected to the cassette body 201, e.g. to the fluid supply chamber 3, with the aid of fixation devices 15, for example rivet connections, weld seams or the like.

A fluid connector 17 is connected to the rear support structure 11 of the filter device.

Through the fluid connector 17, a second fluid 19 is introduced into the fluid reception chamber 1. The second fluid may be used for treating the first fluid 5, for example by applying pressure.

As is shown in the FIG. 1, the fluid reception chamber 1 represents a lower region 21 and the fluid supply chamber 3 represents an upper region 23.

The section of the blood cassette 200 shown in FIG. 1 is arranged such that the front side of the filter membrane 9 is viewed. The filter membrane 9 is disposed with its side lying behind the drawing plane on the rear support structure 11. The latter is denoted as “rear” support structure because the rear side faces the filter membrane 9 or is disposed on the rear side of the filter membrane 9.

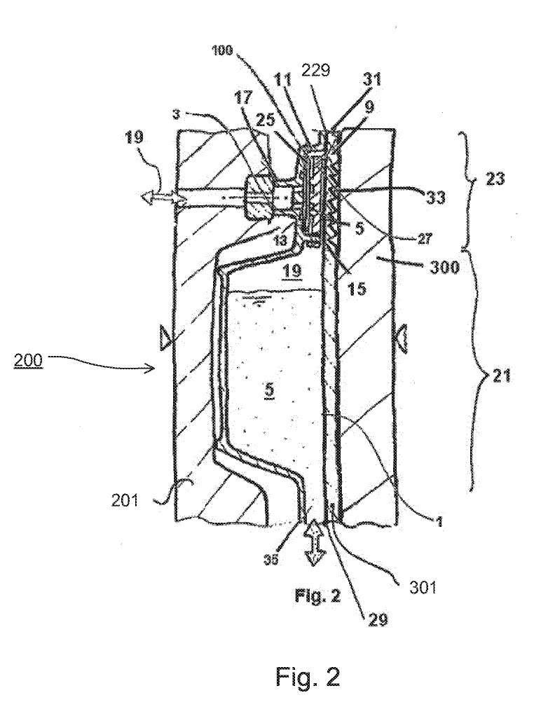

FIG. 2 shows a blood cassette 200 in a cross-sectional view. The blood cassette 200 is pressed with or to a treatment apparatus 300.

The lower region 21 forms the deeper region, i.e., the fluid reception chamber 1. The upper region 23 forms the shallower region, i.e., the fluid supply chamber 3.

As is shown by the double arrow in FIG. 2, a second fluid 19 is introduced into the fluid supply chamber 3 through a fluid connector 17.

The fluid supply chamber 3 optionally comprises, as represented in FIG. 2, the rear support structure 11 which is fastened to the fluid supply chamber 3 by means of a sealing connection 25, for example a welded or bonded connection.

On the rear support structure 11 the filter membrane 9 is disposed, with a front support structure 27 in turn being provided on the latter.

The cassette body 201 of the blood cassette 200 is covered at one side by a cover element 229, for example a film.

By means of or through the intermediary of a rubber mat 301 which may serve for transmitting force and/or movements by sensors or actors of the treatment apparatus 300 to the blood cassette 200 or chambers and/or passages thereof, the blood cassette 200 is pressed with the treatment apparatus 300.

The rubber mat 301 may have a resilience structure 33.

In order to introduce or remove the fluids present inside the fluid reception chamber 1, the fluid reception chamber 1 may be provided with a fluid connection 35 at its bottom side.

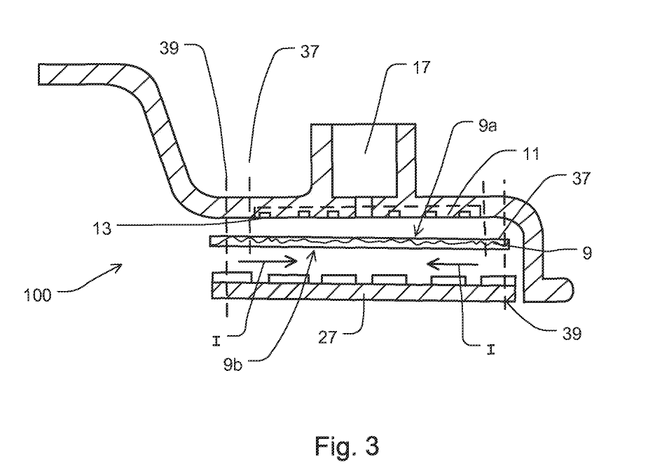

FIG. 3 shows an enlarged detail or section of the blood cassette 200 of FIG. 2. To be more precise, it shows the filter device in a longitudinal sectional view.

In comparison with the representation of FIG. 2, the filter device was tilted by 90 degrees, so that the fluid connector 17 is directed upwardly in the representation of FIG. 3.

The tilting serves a better representation. During use of the blood cassette 200 the filter device takes or maintains or keeps its position of FIG. 1.

The filter membrane 9 which exemplarily comprises a membrane layer and a third support layer (not represented) in the form of a woven, is disposed between the rear support structure 11 and the front support structure 27.

For a detailed description of the single components, reference is made to the above explanations or embodiments.

In order to establish the sealing connection between the rear support structure 11 and the filter membrane 9, an optional first ring zone or first ring region 37 is provided. In order to establish the connection between the filter membrane 9 and the front support structure 27, an optional second ring zone or second ring region 39 is provided.

The arrows I of FIG. 3 show or point towards the inside or central section of the filter membrane 9. They point from the edge of the filter membrane 9 towards the inside.

The reference numeral 9a designates the rear side of the filter membrane 9, the reference numeral 9b designates the front side of the filter membrane 9.

The filter membrane 9 may have a carrier fleece on its rear side 9a and/or on its front side 9b. A carrier fleece may be a woven, it may comprise a Teflon layer.

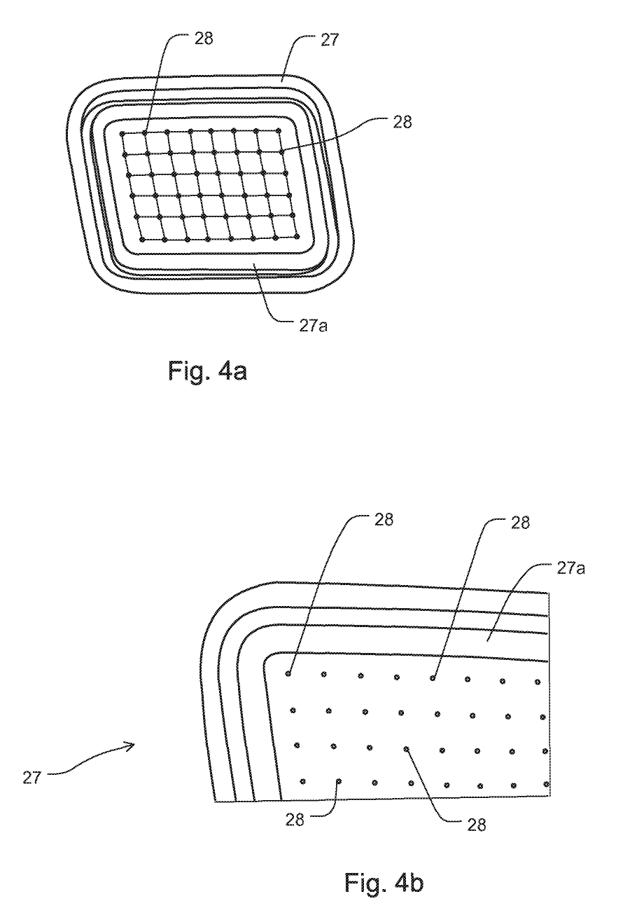

FIG. 4a shows the front support structure 27 with a first contact section 27a. The illustration of FIG. 4a corresponds to the view of the side of the front support structure 27 which in turn faces, during use, the front side 9b of the filter membrane 9. The front support structure 27 comprises openings 28 passing through it, which are explained below with reference to FIG. 4b.

As is exemplarily shown in FIG. 4a, the first contact section 27a may be made of another or different material than the remaining sections or sections of the front support structure 27. For example, the first contact section 27a may be made of or may comprise silicon and may optionally be inserted for example as an O-ring into a groove or into a slot of the front support structure 27. Hence, the front support structure 27 may be substantially made of comparatively favorable or low-priced and harder material. At the same time, its first contact section 27a may be made of a softer material which may, although more expensive when compared to the harder material, advantageously cause no damage, unlike the harder material, when the first contact section 27a contacts the filter membrane 9 and when the front support structure 27 is being pressed against the filter membrane 9.

The first contact section 27a may, unlike what is shown in FIG. 4a, alternatively be made of the same material as the remaining sections or other sections of the front support structure 27. For example, the entire front support structure 27 including the first contact section 27a may be made of or may consist of silicon. This offers production advantages at rather higher material cost, as silicon is not a cost-effective material.

Some aspects as described herein encompass further embodiments by which, unlike the illustration of FIG. 4a, all sections of the front support structure 27 are made of, or consist of, a material, which material is not silicon. In this way, the front support structure 27 may be manufactured at low cost. The desired low hardness of the front support structure 27 is achieved when or upon connecting the front support structure 27 to the cassette body 201 during welding and due to welding the front support structure 27 in the area of the first contact section 27a to the cassette body 201.

FIG. 4b shows an enlarged detail of the front support structure 27 of FIG. 4a.

Openings 28 are shown, which make the front support structure 27 permeable for fluid which also can flow through the filter membrane 9. The permeability of the front support structure 27 for the fluid may optionally be rendered possible alone by the openings 28 as in the present example.

The diameter of (all or many of) the openings 28 comprises preferably a maximum of 0.7 mm, preferably not more than 0.5 mm. If the openings 28 are configured as slots, the previous measurements or dimensions apply analogously for the width of the slots.

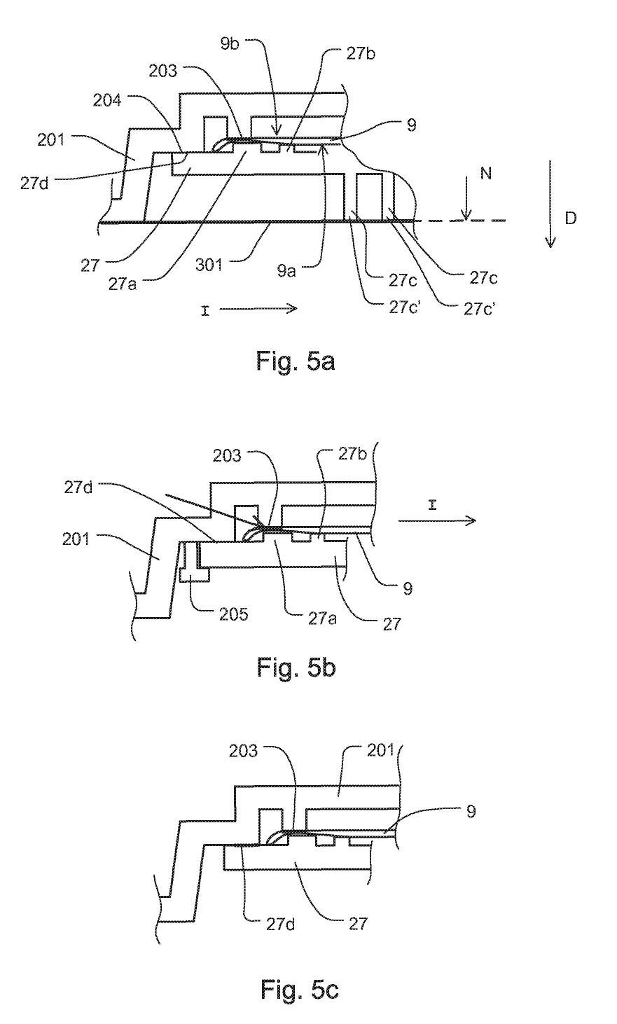

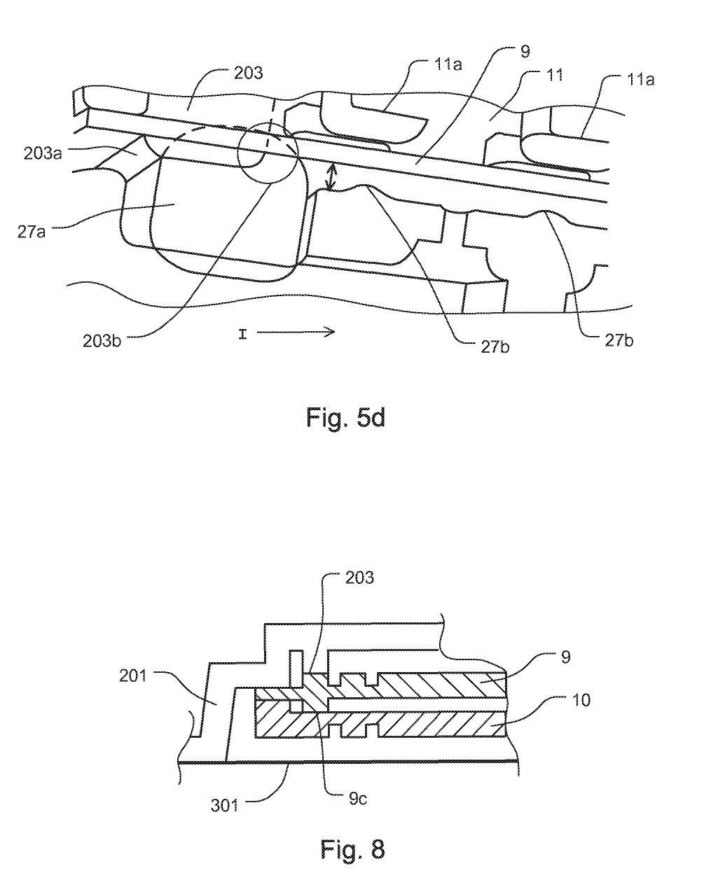

FIG. 5a shows a schematically highly simplified detail of an exemplary embodiment of the blood cassette 200 having the cassette body 201 (as in FIGS. 5b to 5d shown in 180° side rotation relative to its illustration in FIG. 3), a filter membrane 9, a front support structure 27 and a rubber mat 301. The rubber mat 301 is part of a blood treatment apparatus 300, not shown in FIG. 5a, into which the blood cassette 200 is received.

The filter membrane 9 is welded to the cassette body 201 along a weld section 203. The weld section 203 may be circumferentially closed. This is not seen in FIG. 5a due to the cut illustration of this figure. The weld section 203 may be understood as the first ring region 37, see FIG. 2.

The front support structure 27 rests with a first contact section 27a at least partially or in-section on the weld section 203 or contacts the latter.

Preferably, the first contact section 27a extends or stands over the weld section 203 towards a direction I, i.e. towards the central section of the front support structure 27 or the filter membrane 9. This is shown and explained further in an enlarged view in FIG. 5d.

In the example of FIG. 5a, the front support structure 27 optionally comprises burls or protrusions 27b which preferably protrude or rise from the side of the front support structure 27 on which the front support structure 27 of the filter membrane 9 rests or with which it faces the filter membrane 9.

The burls or protrusions 27b are arranged on the edge side and/or in a middle area of the front support structure 27. They comprise slots in between so that a gas exchange or flow through said slots is rendered possible.

In the example of FIG. 5a the front support structure 27 comprises one or several burls or protrusions 27c, which preferably protrude from the side of the front support structure 27 which lies opposite to the side of the front support structure 27 which faces the filter membrane.

The rubber mat 301 further shown in FIG. 5a presses on the burls 27c of the front support structure 27.

The burls or protrusions 27c protrude preferably to a pre-determined extent in direction D. The direction D is perpendicular to the main extension plane of the filter membrane 9 and/or perpendicular to the main extension plane of the front support structure 27. This extent may be pre-determined such that the end sides 27c′ reach the rubber mat 301. This extent may be pre-determined such that the end sides 27c′ reach to a level N at which the cassette body 201 is covered in direction D with a film which is not shown in FIG. 5a.

In the example of FIG. 5a, the front support structure 27 optionally comprises a second contact section 27d. The second contact section 27d rests on a support section 204. The support section 204 may be configured as a protrusion or edge of the cassette body 201.

The support section 204 and/or the second contact section 27d may be circumferentially closed. This is not seen in FIG. 5c due to the cut illustration of this figure.

The first contact section 27a is disposed, with respect to the direction I, further inside than the second contact section 27d.

FIG. 5b shows a schematically highly simplified detail of the blood cassette 200 again in a further exemplary embodiment having the cassette body 201, a filter membrane 9, a front support structure 27 and a flanging or a flange edge 205. The flange edge 205 serves the clamping of the front support structure 27 at the cassette body 201 and in particular the pressing between the first contact section 27a and the weld section 203.

FIG. 5c shows a schematically highly simplified detail of the blood cassette 200 again in a further exemplary embodiment having the cassette body 201, a filter membrane 9, a front support structure 27 and a weld connection between the second contact section 27d of the front support structure 27 and the support section 204 of the cassette body 201.

The weld connection between the second contact section 27d and the support section 204 may be circumferentially closed. This is not seen in FIG. 5c due to the cut illustration of this figure.

FIG. 5d shows in an enlarged illustration that the first contact section 27a contacts or touches the weld section 203 as well as parts of the weld seam 203a, by means of which weld seam 203a the first contact section 27a contacts or is connected to the weld section 203.

It is also seen that the first contact section 27a comprises a section 203b which extends or stands over the weld section 203 in—herein exemplarily exactly one direction I. The latter is an optional feature already shown in FIG. 5a to FIG. 5c.

A free space between the burls or protrusions 27b of the front support structure 27 on one hand and the filter membrane 9 on the other hand should not exceed 0.5 mm, preferably 0.3 mm. This space is indicated in FIG. 5d by double arrow. If it is larger than indicated supra, the filter membrane 9 may be too strongly bent before it comes to lie on the burls or protrusions 27b of the front support structure 27. A result thereof could be an undesirable high mechanical stress.

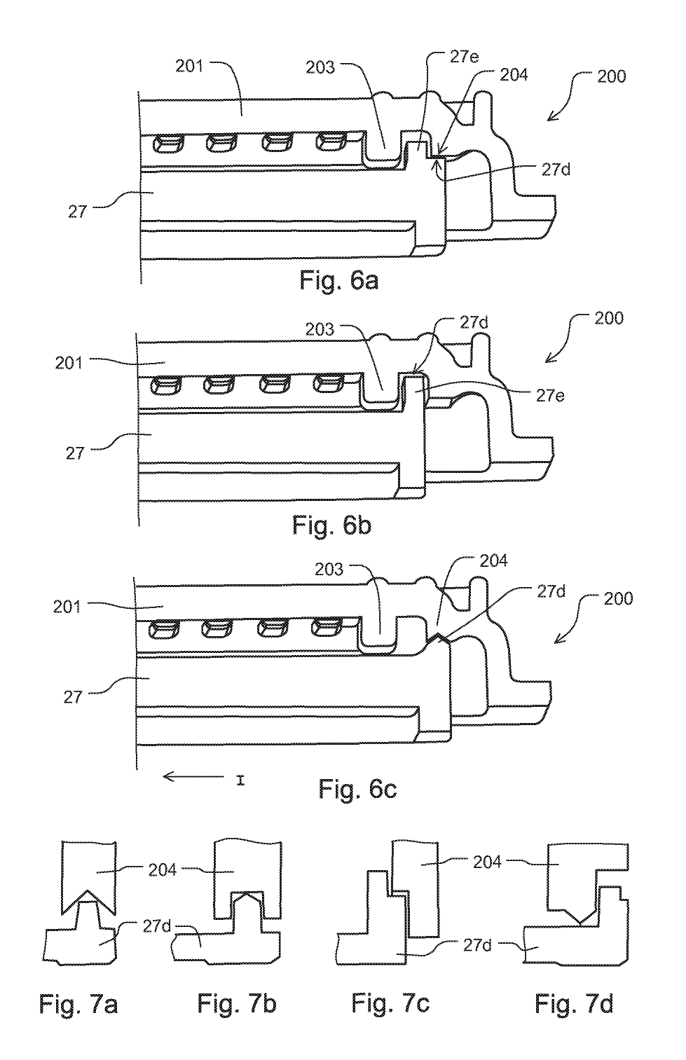

FIGS. 6a to 6c show sections of both the cassette body 201 and the front support structure 27 in diverse embodiments of the blood cassette 200.

The filter membrane 9 is not shown in FIGS. 6a to 6c, wherefore the weld section 203 cannot be shown in a welded state in these figures either.

The second contact section 27d is embodied in FIG. 6a as a closed surface which plane is or extends parallel to a main extension plane of the filter membrane 9, which is not shown, or to the main extension plane of the front support structure 27, like in the example shown in FIG. 5a.

A preferably likewise closed collar or edge 27e, whose inner and/or outer side may be perpendicular to the closed surface of the second contact section 27d, protrudes or rises on an inner side (left in FIG. 6a, see the arrow I). The edge 27e may be considered as a centering aid when inserting the front support structure 27 into the cassette body 201. It may serve when holding the front support structure 27, while the latter is being welded to the cassette body 201.

Although the edge 27e in FIG. 6a rests on an inner side of the second contact section 27d, it is intended to also alternatively provide it on the outer side of the second contact section 27d.

In FIG. 6b, the second contact section 27d and the protruding edge 27e merge into each other; the second contact section 27d may be considered as an end section of the protruding edge 27e.

In FIG. 6c, the second contact section 27d and the support section 204 of the cassette body 201 shown in a cross section in FIG. 6c are not flat, but curved, triangle or polygonal. This may support a centering effect. Moreover, an area or surface advantageously rises or elevates (ceteris paribus); said area being available for a contact between contact section 27d and the support section 204, e.g. for a welding. The aforementioned advantages may also be achieved when only the second contact section 27d or the support section 204 of the cassette body, but not both, are formed as described supra. Examples thereof are found in the following figures.

FIGS. 7a to 7d show different embodiments of the second contact section 27d and the support section 204 of the blood cassette 200.

FIG. 7a and FIG. 7b show embodiments which correspond to or resemble the embodiment according to FIG. 6c.

FIG. 7c shows an embodiment which corresponds to or resembles the embodiment according to FIG. 6a.

FIG. 7d shows an embodiment which corresponds to a combination of the embodiments according to FIGS. 6b and 6c.

FIGS. 7b and 7c thereby show embodiments which are particularly suitable in the light of shrinking during welding.

FIG. 8 shows a second filter membrane 10 which is provided instead of or in addition to the support structure 27.

The filter membrane 9, which in some exemplary embodiments could be referred to also as first filter membrane for better distinction, comprises a weld section or a welding 9c by means of which it is connected to a second filter membrane 10.

The second filter membrane 10 may be configured as the filter membrane 9 with regard to the features which contribute to filtering. This is optional, i.e. not necessarily required.

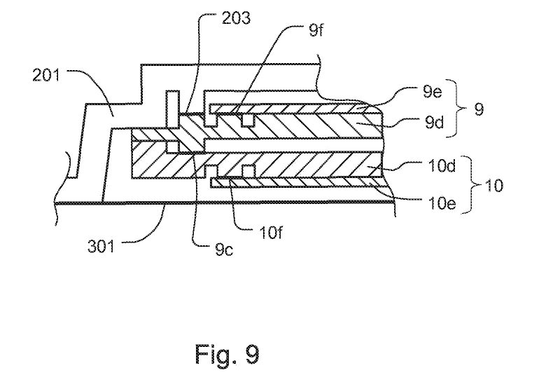

FIG. 9 shows as FIG. 8 a second filter membrane 10 which is provided instead of or in addition to the front support structure 27.

Both the filter membrane 9 and the second filter membrane 10 are divided into or comprise each a carrier fleece 9d or 10d, respectively and a Teflon layer or coating 9e or 10e, respectively.

Thereby, the filter membrane 9 and the second filter membrane 10 are arranged such that their carrier fleece 9d and 10d face each other, respectively. The Teflon layers 9e and 10e rest on the side of the carrier fleece 9d or 10d, respectively which faces away from the other filter membrane 10 or 9, respectively. In other words, the Teflon layers 9e or 10e thus point towards the “outside”, respectively.

In addition to the welding 9c of the filter membrane 9 with the second filter membrane 10 and the weld section 203 which are both known from FIG. 8, further two optional weld sections 9f and 10f are shown in FIG. 10.

Weld seam section 9f connects the carrier fleece 9d to the Teflon layer 9e by means of welding. Weld seam section 10f connects the carrier fleece 10d to the Teflon layer 10e by means of welding.

Weld seam section 9f may be provided in a closed manner like weld seam section 10f.

The Teflon layer 9e or 10e may each be made of, or comprise, PTFE (Polytetrafluoroethylene) or ePTFE (expanded Polytetrafluoroethylene).

FIG. 10 shows a further embodiment of the front support structure 27. This front support structure 27 comprises a profiled structure 29 in its area facing the weld section 203. The profiling 29 may be directed to the interior of the support structure 27 perpendicularly or almost perpendicularly or may extend such that channels are formed between the raised profiles. Such profiling 29 (combination between raised structures on the one side and channels on the other) may effect that when melting the structure for fastening the filter membrane to or on the front support structure 27, the material does not penetrate or only slightly penetrates into the central area of the filter membrane, but predominantly spreads or extends into or within the channel areas.

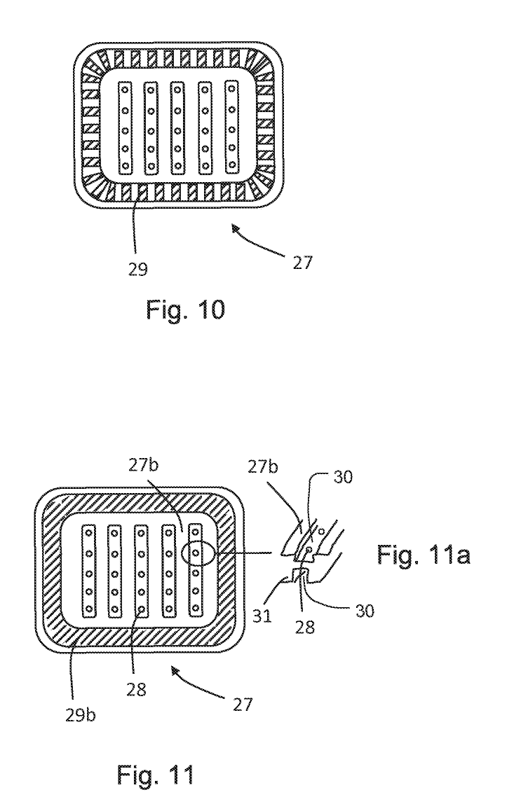

This is schematically shown in FIG. 11, where the material of the raised structures (shaded area of FIG. 10) extends into or within the area of the channels. The embodiment of FIG. 11 of the front support structure 27 may also be achievable by at least a partial melting of the raised portions of the front support structure 27 of FIG. 10. With that, the profiling 29 may at least be partially recognizable in the melted profiling 29b. The front support structure 27 may, deviating from the illustration of FIG. 11, be also designed as parallelogram.

The profiling 29 may be provided in a closed manner in the front support structure 27. This front support structure 27 may be designed as one-piece or may consist of one material. It has become evident that this embodiment may be suitable, even without additional softer support elements, to support the filter membrane which is sterilized by rays or e-beam so that a delamination of the filter membrane can be avoided.

Even though the following features are described with respect to the embodiment of FIG. 11, they may however be realized in all other embodiments as described herein as well. The front support structure 27 may comprise openings 28. These may be arranged in recesses 30 such that burls or protrusions 27b may be flatly formed between the openings 28. In the area of the openings 28, the front support structure 27 may also be formed thinner than in other areas. The principle is shown in FIG. 11 in the encircled areas in a front cut oblique view. Thus, the front support structure 27 may comprise in addition to the flat burls or protrusions 27b, which face the filter membrane, a profiling 31 on the side which faces away from the filter membrane. The thinner design of the support structure 27 in the area of the openings may allow gas to pass through easier (lower flow resistance); the additional profiling 31 may effect a stabilization by or in the form of higher bending strength.

All of the front support structures 27 and blood cassette bodies described above may comprise, or may consist of, polypropylene.

FIG. 11a shows an enlarged detail of FIG. 11.

### LIST OF REFERENCE NUMERALS

1 fluid reception chamber
3 fluid supply chamber
5 medical fluid
7 fluid level
9 filter membrane
9a rear side of the filter membrane
9b front side of the filter membrane
9c welding with a second filter membrane
9d carrier fleece
9e Teflon layer or coating
9f weld seam section
10 second filter membrane
10d fleece carrier
10e Teflon layer
10f weld seam section
11 rear support structure
11a burls or protrusions
13 drainage structure
15 fixation devices
17 fluid connector
19 second, gaseous fluid
21 lower region
23 upper region
25 sealing connection
27 front support structure
27a first contact section of the front support structure
27b burls or protrusions
27c burls or protrusions
27c′ end sides
27d second contact section
27e edge, collar
28 openings
29 profiling/profiled structure
29b melted profiling
30 recesses
31 profiling/profiled structure
33 resilience structure
35 fluid connection
200 blood cassette as an example of a medical device
201 cassette body or cassette main body, hard part
203 weld section
203a weld seam
203b section
204 support section
205 flanging or flange edge
229 cover element, film
300 blood treatment apparatus
301 rubber mat
D direction
I direction
N level

1.-16. (canceled)

17. A medical device comprising:
a fluid system for receiving a first medical fluid;
a device body; and
at least one hydrophobic filter device with a filter surface through which at least a second gaseous fluid may be supplied to the fluid system, wherein the filter device comprises:
at least one filter membrane having a front side and a rear side; and
a front support structure at the at the front side of the filter membrane,
wherein the rear side of the filter membrane is welded to a section of the device body along a weld section, and
wherein the front support structure is arranged to contact the weld section through a first contact section of the front support structure.

18. The device according to claim 17, wherein the front support structure comprises a second contact section, wherein the front support structure is arranged to contact a support section of the device body through the second contact section of the front structure.

19. The device according to claim 18, wherein the second contact section is welded to the support section of the device body.

20. The device according to claim 19, wherein the weld section is a circumferential or closed section.

21. The device according to claim 19, wherein the first contact section is a circumferential or closed section.

22. The device according to claim 18, wherein the second contact section is clamped with the support section of the device body.

23. The device according to claim 17, wherein the first contact section of the front support structure is made of a softer material than other sections of the front support structure.

24. The device according to claim 17, wherein the front support structure is made of exactly only one material.

25. The device according to claim 17, wherein the first contact section of the front support structure protrudes or extends beyond the weld section of the device body in at least one direction or protrudes or extends at one side of the weld section.

26. The device according to claim 17, wherein the first contact section of the front support structure is connected to sections of the front support structure in a releasable manner.

27. The device according to claim 17, wherein the first contact section of the front support structure differs in color from sections of the front support structure.

28. The device according to claim 17, wherein the front support structure comprises one or more openings having a maximum diameter or width of 0.7 mm.

29. The device according to claim 17, wherein the front support structure comprises one or more openings having a maximum diameter or width of 0.5 mm.

30. The device according to claim 17, wherein the front support structure comprises a second filter membrane.

31. The device according to claim 17, wherein the front structure is a second filter membrane.

32. The device according to claim 17, wherein the medical device is a blood cassette.

33. The device according to claim 17, wherein the first contact section of the front support structure comprises a section made or formed by melting or temporary liquefying material during manufacturing of the device.

34. The device according to claim 17, wherein the first contact section of the front support structure is a section made or formed by melting or temporary liquefying material during manufacturing of the device.

35. A method for manufacturing a medical device, the method comprising:
providing a device body of a medical device with a fluid system for a first medical fluid;
providing a filter device with a filter surface through which a second gaseous fluid may be supplied, wherein the filter device comprises at least one filter membrane having a rear side and a front side, wherein the rear side of the filter membrane is welded to a section of the device body along a weld section;
providing a front support structure;
arranging the front support structure on or at the device body on the front side of the filter membrane such that it contacts the weld section through a first contact section of the front support structure; and
welding together the front support structure and the front side of the filter membrane.
